(19) United States
(12) Patent Application Publication
(10) Pub. No.: US 2011/0313299 A1
(43) Pub. Date: Dec. 22, 2011

(54) SEVERAL MEASUREMENT MODALITIES IN A CATHETER-BASED SYSTEM

(75) Inventor: James F. Brennan, III, Matthews, NC (US)

(73) Assignee: Prescient Medical, Inc., Doylestown, PA (US)

(21) Appl. No.: 13/224,988

(22) Filed: Sep. 2, 2011

Related U.S. Application Data
(63) Continuation of application No. 12/112,916, filed on Apr. 30, 2008.
(60) Provisional application No. 60/915,085, filed on Apr. 30, 2007.

(51) Int. Cl. A61B 6/00

(52) U.S. Cl. 600/478

(57) ABSTRACT

The invention provides optical switching-based systems and methods for catheter-based optical diagnosis which are particularly well suited to determining the condition of blood vessels, including the state of the vessels with respect to atherosclerosis and its development. Various embodiments provide catheter-based spectroscopic systems that are configured to cycle between different optical interrogation techniques such as Raman spectroscopy, optical coherence tomography and time-resolved laser-induced fluorescence spectroscopy. One embodiment of the invention provides an intravascular catheter-based diagnostic system that cycles between fingerprint region (200-2,500 cm⁻¹) Raman spectroscopy and high-wavenumber region (2,500-4,000 cm⁻¹) Raman spectroscopy.

### CROSS-REFERENCE TO RELATED APPLICATIONS

This application is a continuation of U.S. patent application Ser. No. 12/112,916 filed Apr. 30, 2008, which claims the benefit of U.S. Provisional Application No. 60/915,085 filed Apr. 30, 2007, which is incorporated by reference herein in their entirety.

### FIELD OF THE INVENTION

This invention relates to spectroscopic apparatuses that are configured to switch or cycle between multiple spectroscopic techniques.

### BACKGROUND OF THE INVENTION

During the past twenty years, the use of catheters to enter, diagnose, and treat diseases and malfunctions of the blood vessels and other vessels has become commonplace. Catheters are widely employed to deliver stents to occluded blood vessels, as well as to position and deploy balloons to enlarge occluded blood vessels. Also, catheters are used in combination with high power lasers for treating and removing atherosclerotic plaque via an optical removal process called ablation.

In certain situations, medical professionals have been unable to take advantage of these relatively non-invasive catheters. For example, in the case of totally occluded vessels, it is difficult or impossible to safely insert and position guidewires and catheters for stent deployment. In hundreds of thousands of these cases per year, open-heart surgery is necessary to treat these patients, which in addition to a long and painful recovery and high expense, carries significant risks.

Similarly, the usefulness of catheters in treating and removing plaque is often limited. Recent findings indicate that some nonstenotic, or nominally stenotic, lipid-rich coronary plaques with thin luminal coverings, also called “vulnerable plaques” or “biological hot plaques,” are exceptionally likely to cause the vast majority of fatal heart attacks. The majority of the approximately 1,300,000 heart attacks that will occur this year are caused by these lipid-rich plaques (“soft plaques”).

Various tests exist for identifying persons at risk of myocardial infarction. Once they are identified, these persons are candidates for further evaluation and treatment. An ideal treatment system would allow for the use of multiple devices within a single catheter, therefore allowing several functions, some complementary, over the course of a single catheter insertion procedure. For instance, one such system which would allow: a) the use of an optical interferometer capable of determining structural information about an arteries wall; b) the use of a spectroscopy system that uses multiple wavelengths of light to differentiate among various materials in the optical path, including vulnerable plaque, calcified plaque, arterial walls, etc.; and c) the intermittent use of an laser to ablate, vaporize, or otherwise destroy the plaque in the select regions in the path of the catheter.

Several types of optical instruments routinely are used in catheter insertion procedures. For example, optical interferometry of various types that typically employ Michelson interferometers, such as Optical Coherence Tomography (OCT) and low coherence interferometry (LCI), are used to differentiate between plaque and arterial walls with spatial resolution in the range of ˜10 micrometers. Also, optical spectroscopies, such as Diffuse Reflectance Near Infrared Spectroscopy (DRNIRS), Raman spectroscopy, and fluorescence spectroscopy, have been shown to effectively differentiate and identify a wide variety of substances, such as glucose, calcified plaque, lipid-rich plaque, proteins, human metalloproteins, creatinine, uric acid, triglycerides, etc. Most forms of spectroscopy provide the capability to detect and determine materials without contacting or touching them. Substances are distinguished by their unique optical absorption, scattering, and reflectance properties, which is specific to differing wavelengths of light. Another optical modality used in catheter procedures involves ablation lasers that use very short light pulses, typically less than 1 microsecond in duration but may be less than 1 picosecond in duration, to remove tissue.

Other devices for evaluating and treating arterial disease are known to those skilled in art. As with all optical devices, it is generally known to use either a single fiber or a bundle of fibers to transmit one or more optical signals. Often these devices are intended to improve the spatial resolution and/or information available to a clinician by using a single measurement modality. Examples of such uses are described in the following U.S. patents. U.S. Pat. No. 5,217,456, issued to Narciso, Jr., discloses a catheter for ablation of a lesion. The rotating catheter has a bundle of optical fibers that are used to make fluorescence measurements to identify the radial position of the lesion. U.S. Pat. No. 6,384,915, issued to Everett, et al., and U.S. Pat. No. 6,175,669, issued to Colston, et al., disclose the use of a multiplexed reflectometer for performing interferometry. Both patents describe a system including an optical fiber set contained within the catheter. The optical fibers are connected to the illumination source via an optical switch, which sequentially cycles the output of the source through the optical fiber set to diagnose consecutive spatially-distinct regions of a lumen. U.S. Pat. No. 6,463,313, issued to Winston, et al., describes a device having dual Michelson interferometers. The outputs are combined to produce a composite image thereby providing more complete information to medical practitioners. U.S. Pat. No. 6,501,551, issued to Tearney, et al., discloses the combination of two sources of differing wavelengths by using wavelength division multiplexing. The combined signal is injected into a single optical fiber in the catheter. The reflections are separated by wavelengths and guided to separate detectors associated with a particular wavelength.

Devices combining some navigation or diagnostic element, such as fluorescence spectroscopy, with a treatment element, such as an ablation laser, are known to those skilled in the art. These devices are represented by the angioplasty systems such as those described in U.S. Pat. No. 5,275,594, issued to Baker, et al. and in U.S. Pat. No. 6,463,313, Winston, et al. Both Baker, et al., and Winston, et al., disclose systems that use feedback from the diagnostic element to control the operation of the treatment element. U.S. Pat. No. 6,389,307, issued to Abela, discloses a system having a lower power diagnostic laser and a high power treatment laser coupled to the same optical fiber. The operator activates the desired laser, preferably one at a time, to achieve a desired function.

U.S. Publication No. 20060139633 discloses methods and systems of high-wavenumber Raman spectroscopy for measuring tissue properties including for characterizing atherosclerotic plaques, and is incorporated by reference herein in its entirety.

U.S. Pat. No. 6,272,376 discloses methods and systems of time-resolved laser-induced fluorescence spectroscopy, including for identifying and characterizing lipid-rich vascular lesions, and is incorporated by reference herein in its entirety.

International Publication No. WO2005019800 discloses methods for fluorescence lifetime imaging microscopy and spectroscopy, including ultra-fast methods for analysis of fluorescence lifetime imaging is also described, facilitating real-time analysis of compositional and functional changes in samples, and is incorporated by reference herein in its entirety.

Low-coherence interferometry methods, such as OCT, are disclosed in U.S. Pat. Nos. 7,190,464, 6,903,854 and 6,134,003, each of which is incorporated by reference herein in its entirety.

U.S. Pat. No. 7,061,606 and U.S. Pub. No. 20040077950 disclose near-infrared (NIR) spectroscopy, such as analysis of NIR absorbance, transmittance and reflectance spectra, and are incorporated by reference herein in their entireties.

U.S. Pub. No. 20020183601 discloses laser speckle-based methods and systems for analyzing tissue, and is incorporated by reference herein in its entirety.

An optical switching system for use with an catheter system that combines optical diagnostic and/or treatment so that these measurements can be performed during a single catheter insertion would offer dramatic benefits to save lives and preclude coronary events. This procedure would be an effective, efficient, and safe method for treating a very dangerous condition, especially when compared to the options of performing no procedure or performing bypass surgery.

### BRIEF SUMMARY OF THE INVENTION

An apparatus and method for treatment of the arteries of the heart using optical switches to allow safe navigation of blood vessels with a catheter through the use of one or more interferometer systems and intermittent or concurrent treatment through the use of a treatment laser, precise insertion of a stent or other prosthesis to cover the vulnerable plaques, or other tool. The apparatus and method allows differentiation among arterial walls, calcified plaque, vulnerable plaque, such as biological hot plaque, thin capped fibrous atheromas (TCFAs), and other forms and substance in blood vessels. The device and method is useful in the treatment of cardiovascular disease, the performance of hemodialysis access maintenance, and the insertion of Trans-Jugular Intrahepatic Portosystemic Shunts.

The apparatus allows multiple optical sources to be switched into one or more optical fibers in the catheter. The return signal from the catheter is switched between one or more optical detectors, such as an interferometer, a spectrum analyzer, and a reflectometer. The use of optical switches allows the use of one or more interferometric systems and/or types of spectroscopy in the same fiber, as well as controlling the duty cycle of an interrogation method to, say, limit the overall exposure of a given artery wall location to a particular radiation, or switching through several light sources in order to determine geometry and composition in the path of the catheter with various types of interferometry and/or spectroscopy.

The use of an optical switch provides the capability to sample multiple wavelengths and/or ranges of an optical spectra through a single fiber and from the loci of a single fiber end in the catheter into the loci of a single point on an artery wall, quickly enough to safely assure that all the sampling of wavelengths or bandwidth spectra occurred in the same loci in the artery. Interrogation of a single physical location in an artery by multiple optical techniques will provide a medical practitioner with a plethora of information useful in differentiating among artery wall, calcified plaque, hot plaque and other materials.

### BRIEF DESCRIPTION OF THE SEVERAL VIEWS OF THE DRAWINGS

The above-mentioned features of the invention will become more clearly understood from the following detailed description of the invention read together with the drawings in which:

FIG. 1 illustrates one embodiment of an optical switching system for use with catheter-based analysis and treatment;

FIGS. 2A and B illustrates an embodiment of an intravascular catheter adapted for use with the present invention;

FIG. 3 illustrates an intravascular catheter embodiment of the present invention in the environment of an artery;

FIG. 4 illustrates an alternate embodiment of the optical switching system for use with catheter-based analysis and treatment incorporating a treatment laser;

FIG. 5 illustrates an alternate embodiment of the optical switching system for use with catheter-based analysis and treatment adapted for using external optical sources and detectors;

FIG. 6 illustrates an alternate embodiment of the optical switching system for use with catheter-based analysis and treatment adapted for using external optical sources and detectors and incorporating a treatment laser;

FIG. 7 is a flow chart of one method of sequencing the source switch and detector switch in relation to the catheter switch;

FIG. 8 is a flow chart of an alternate method of sequencing the source switch and detector switch in relation to the catheter switch;

FIG. 9 shows Raman spectra of cholesterol and cholesterol esters collected using free space optics;

FIG. 10 illustrates one embodiment of an optical switching system for catheter-based optical analysis; and

FIG. 11 illustrates one embodiment of an optical switching system for catheter-based optical analysis.

### DETAILED DESCRIPTION OF THE INVENTION

An optical switching system for use with catheter-based analysis and treatment, or optical switching system, is shown and described. The use of an optical switching system allows the use of one or more interferometric and/or spectroscopic systems in the same fiber, as well as controlling the illumination duty cycle to, say, limit the overall exposure of a given artery wall location to a particular radiation, or in switching through several light sources in order to determine geometry and composition of the arterial wall in the path of the catheter with various types of interferometry and/or spectrometry.

The use of optical switches greatly aids in safely constructing and using a device for locating, identifying, and removing a blockage. For example, an optical switch facilitates DRNIRS by enabling multiple wavelengths to be launched into one or more optical fibers by rapidly switching among the available wavelengths. It is important to note that while various optical techniques may often be performed on a single type of fiber, for the most part they cannot occur simultaneously as they would interfere with the functionality and resolution of the various interferometers. In the case where multiple optical measurement modalities are useful, the optical switch permits one or more techniques to be performed through the same optical fibers in a catheter. For example, the procedure may use optical coherence tomography (OCT), such as Time-Domain OCT and/or Frequency Domain OCT (FD-OCT), for identifying a thin capped atherosclerotic lesion and also a spectroscopy technique, such as Diffuse Reflectance Near Infrared Spectroscopy (DRNIRS) or Raman spectroscopy, for differentiating between blood, water, vulnerable plaque, calcified plaque, and other objects. Optical ranging techniques, such as OCT and LCI, are typically implemented with a Michelson interferometer, while optical spectroscopy typically requires several optical wavelength to be analyzed to identify different materials. The optical switch allows the necessary wavelengths to be switched through the optical fibers. An optical switch also provides the ability to route the returning light from the end of the catheter to multiple detection devices.

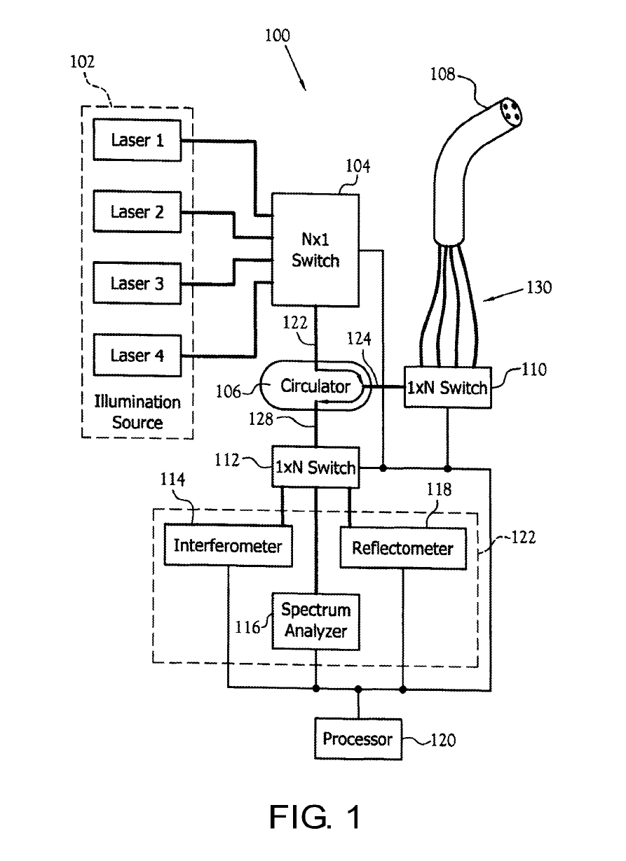

FIG. 1 illustrates one embodiment of a catheter-based analysis and treatment instrument incorporating an optical switching system in a according to the present invention. The medical apparatus includes a multi-wavelength illumination source 102, which may include a low coherence laser, connected optically to a first optical switch 104. In one embodiment, each of the lasers in the illumination source 102 has a unique wavelength and generates a coherent light beam that is useful for navigation of a artery lumen and/or differentiation or identification of objects within the lumen. The first optical switch 104 allows selection of one of the lasers from the bank 102 to be directed through a catheter 108. The first port 122 of a circulator 106, which is optically connected to the first optical switch 104, redirects the selected laser beam through a second port 124 into a second optical switch 110. The second optical switch 110, which is optically connected to the circulator 106, sequentially cycles the selected laser beam through a plurality of optical fibers 130 routed through the catheter 108. The reflections of the laser beam from the catheter 108 are fed back into the circulator 106 through the second port 124 and redirected through the third port 128 of the circulator 106 into a third optical switch 112. The third optical switch 112 connects the returning optical signal to various optical detectors 122. In the illustrated embodiment, the third optical switch 112 is connected to an interferometer 114, a spectrum analyzer 116, and a reflectometer 118. A processing device 120 controls the switching operations for the first optical switch 104, the second optical switch 110, and the third optical switch 112. In addition, the processing device 120 communicates with the optical detectors 122.

As illustrated and described herein, the optical circulator 106 passes signals between successive ports in one direction. Typically, an optical circulator utilizes a nonreciprocal optical element that exploits the optical Faraday effect to spatially separate light propagating in different directions, as is known in the art. However, those skilled in the art will recognize that single direction signal paths can be achieved using other devices, such as wavelength-selective beam splitters in the case of spectroscopy implementations of the invention. The bank of lasers 102 is presumed to have multiple sources; however, those skilled in the art will recognize that a single tunable laser or other tunable source capable of generating the desired wavelengths could be used. In such an arrangement, the single source subsumes the functions of the multiple sources and the first optical switch without departing from the spirit and scope of the present invention. Similarly, the optical detectors 122 are illustrated as including multiple devices performing differing functions. Those skilled in the art will recognize that the optical detectors may include only a single analysis device or single multi-function analysis device and would not require the third optical switch. In either event, such a substitution could easily be warranted by advances in the illumination source or the optical detectors or may merely reflect a medical apparatus performing fewer functions than the illustrated embodiment.

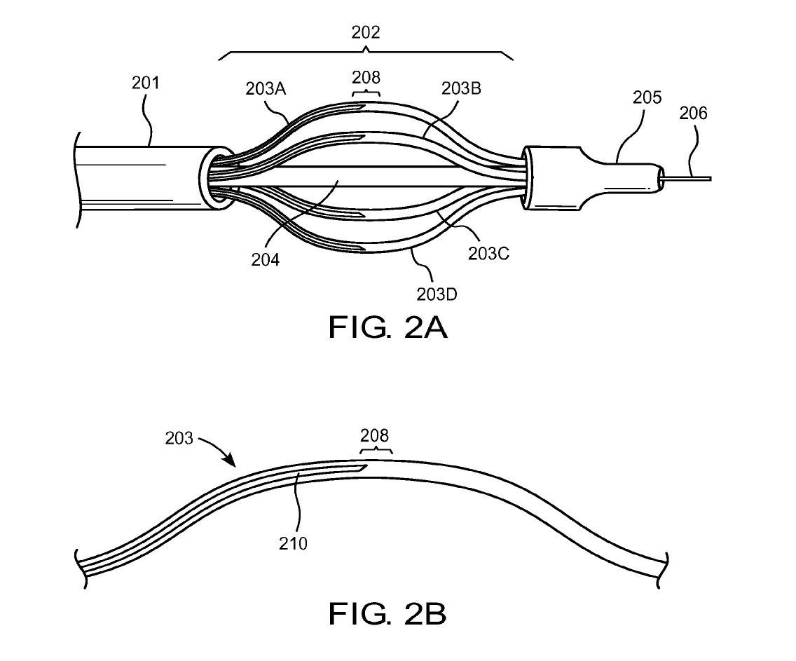

The apparatuses and systems of the present invention may include side/lateral-viewing catheters in which optical components for interrogating the walls of blood vessel lumens are disposed on rods that can be flexed outward toward a blood vessel wall. FIG. 2A shows a basket-style side viewing optical catheter that has four probe arms 203A-D in the basket section 202 of the catheter. Each probe arm may include one or more optical fibers 210 as shown in FIG. 2B that terminate in or around the apex of the radially extended probe arm, i.e., defining a viewing portion 208, to contact or near a vessel wall so that a spectroscopic evaluation of the vessel wall can be performed. The probe arms may, for example, be composed of a polymer, such as a fluoropolymer, such as aTeflon. An optical window may be provided to permit light to transit from the optical fibers to and from a target if the material of the probe arm is insufficiently transparent or provides an undesirable level of interefering background. Windowless probe designs may also be used such as those disclosed in U.S. Ser. No. 11/876,899, which is incorporated by reference herein in its entirety. Each probe arm may also include a structural support element such as spring wire or durable polymer rod (not shown) to support the enclosed fiber optic probe. The catheter also includes a distal tip 505 that is connected to a guidewire tube 504, so that the catheter may travel over a guidewire 506, and connected to the distal end of each probe arm. The proximal end of each probe arm is attached to the proximal end of the basket section of the catheter at or near the distal end of main shaft 201 of the catheter. Radial expansion and contraction of the probe arms of the basket section may be accomplished by contracting and extending the opposite ends of the probe arms, respectively. The guidewire tube, which is attached to the distal tip of the catheter, may be slideable within the catheter thereby permitting said contracting and extending of the opposite ends of the probe with respect to each other. Optional radiopaque marker bands may also be disposed on the catheter to aid in visualizing the catheter within a blood vessel.

A related type of catheter that may be part of the apparatuses and systems of the invention for optically interrogating a blood vessel wall, includes: multiple optical probe rod elements (e.g., 2, 3, 4, 6, or 8) along a central shaft of the catheter and extendable radially outward toward a blood vessel wall from an unextended configuration closer to the longitudinal axis of the catheter and an expandable balloon collectively enclosing the multiple rod elements. The rod elements each include an optical assembly for transmitting and receiving light from the vessel wall lateral to the axis of the catheter while the rod-elements contact or are near the wall. Each of the optical assemblies is in optical communication with at least one optical fiber that is in communication with a light source for illuminating the vessel wall and/or a detector for detecting light received from the vessel wall. The optical assemblies of each rod element may be disposed at or around the middle of a rod element or at or around whatever part of a rod element tends to extend most radially outward. Relative motion of the distal ends and proximal ends of the rods may be used to radially flex the rods outward toward a lumen wall and to radially retract the rods toward the catheter axis.

Apparatuses and systems of the invention may include an intravascular catheter, for optically interrogating a blood vessel wall that includes: (1) a rod element portion near the distal end of the catheter comprising multiple rod elements along a central shaft of the catheter and extendable radially outward toward a blood vessel wall from an unextended configuration closer to the longitudinal axis of the catheter, wherein the rod elements each include an optical assembly for transmitting and receiving light from the vessel wall lateral to the axis of the catheter while the rod-elements contact or are near the wall and wherein each of the optical assemblies is in optical communication with at least one optical fiber that is in communication with a light source for illuminating the vessel wall and/or a detector for detecting light received from the vessel wall; and (2) a tip portion of the catheter that extends from the distal end of the rod element portion to the distal end of the catheter, wherein a guidewire conduit or channel extends from within the central shaft of the rod element portion of the catheter distally through the tip portion of the catheter. The guidewire channel or conduit may, for example, open within the rod element portion of the catheter and at or near the distal end of the tip portion of the catheter.

Any suitable sort of side/lateral-viewing optical assembly(ies) may be used and numerous sorts of side viewing optics are known in the art. For example, a 45-deg (or other angle) mirror face or a prism can be used to laterally direct/redirect light from an optical fiber. Similarly, an optical fiber can be provided with an angularly faceted tip to direct and receive light that is off-axis with respect to the fiber.

In addition to side/laterally-viewing windowless fiber optic probes, the present invention may also be readily implemented in end/front-viewing spectroscopic probe embodiments. Accordingly, the catheter of the present invention incorporates multiple optical fibers fed by an optical switch with other medically necessary and/or useful features; however, those skilled in the art will recognize that configuration and features of the catheter depend upon the usage for which the catheter is designed.

Those skilled in the art will recognize that the number of optical fibers depends upon the desired field of vision and the image processing occurring at the analysis device and, therefore, that number can be varied without departing from the scope and spirit of the present invention. Similarly, the arrangement of the optical fibers depends both upon number and the desired field of vision. Typical, the optical fibers will be equidistantly spaced around the perimeter of the primary tube to provide the most complete field of vision; however, those skilled in the art will recognize other arrangements may be used without departing from the scope and spirit of the present invention.

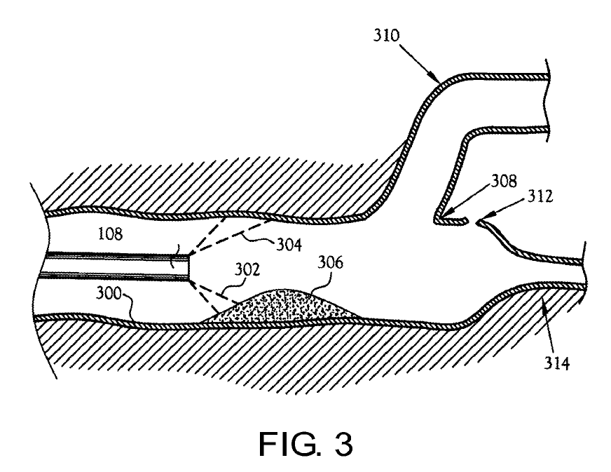

FIG. 3 is a cross-section showing a catheter navigating through a blood vessel 300. The dashed cones represent the upper field of view 302 and the lower field of view 304. The left and right side fields of view are not depicted. In the illustrated embodiment, the blood vessel 300 includes a variety of objects which require navigation or identification. The objects include a bump 306, such as a plaque deposit, a bifurcation 308 of the blood vessel, a turn 310 in the blood vessel, an aortic dissection 312 (or other similar damage to the blood vessel), and a closure or narrowing 314 of the blood vessel.

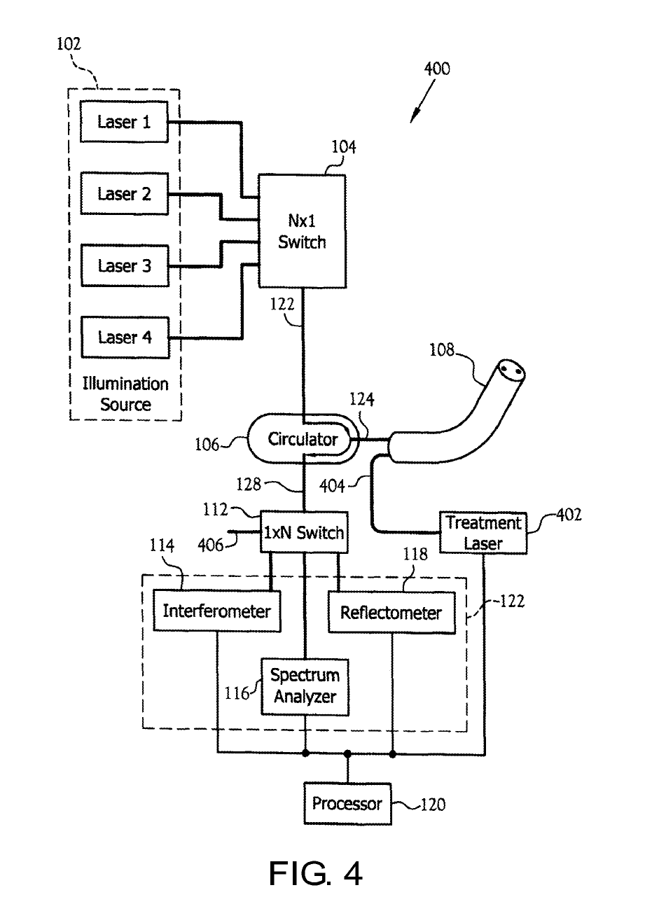

FIG. 4 illustrates an alternate embodiment of a medical apparatus 400 incorporating an optical switching system in a catheter-based analysis and treatment instrument according to the present invention. The medical apparatus 400 includes a treatment laser 402, such as an ablation laser, for example an excimer laser, to reduce an arterial blockage such as a plaque deposit, and/or an infrared laser.(see discussion below) A separate optical fiber 404 in optical communication with the treatment laser 402 runs through the catheter 408. The medical apparatus 400 also includes a shunt 406 that is connected to the optical path during the operation of the treatment laser 402. The shunt 406 is a dead-end optical path where higher power reflectances from the treatment laser 402, which return through the optical fiber 124, are routed to prevent damage to the sensitive interferometry devices 122.

An infrared laser may be used to treat vulnerable plaque lesions or other atherosclerotic lesions and more generally be used to reduce inflammation at a target site of a blood vessel. U.S. Pat. Nos. 6,475,210 and 7,123,968 disclose the use of infrared radiation to treat atherosclerosis and are incorporated by reference herein in its entireties. Instead of a laser, a treatment light source may also be more generally provided. For example, a laser or a LED may be used to provide light to activate a photoactive agent for photodynamic therapy (PDT) of a blood vessel, such as for the treatment of atherosclerosis generally and vulnerable plaque in particular. PDT in the treatment of atherosclerosis is disclosed, for example, in U.S. Pat. No. 6,906,050 and U.S. Pub. Nos. 20060100190 and 20040093044, each of which is incorporated by reference herein in its entirety.

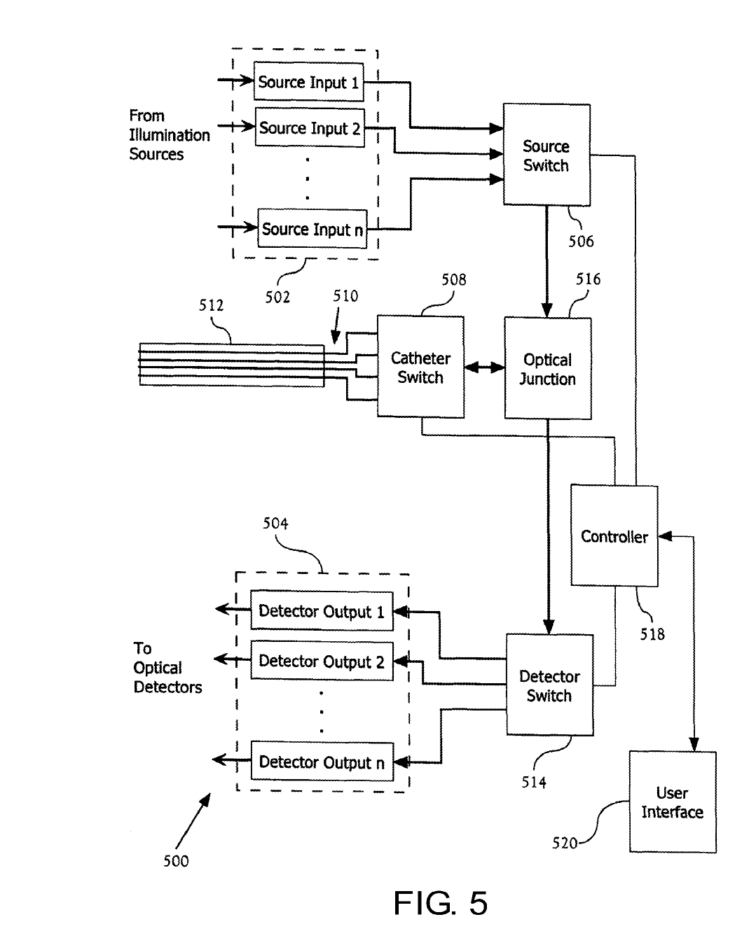

FIG. 5 illustrates yet another embodiment of the medical apparatus 500 adapted for optical navigation and optical identification, for example, to guide non-optical treatments, such as stent insertion or angioplasty. This embodiment of the medical apparatus 500 includes a plurality of input ports 502 for receiving optical signals from external optical sources, and a plurality of output ports 504 for transmitting optical signals to external optical detectors (not shown). The input ports 502 are routed through an optical switch 506. The input port optical switch 506 is optically connected to another optical switch 508 associated with a group of optical fibers 510 carried by a catheter 512. The catheter optical switch 508 is also optically connected to a third optical switch 514 associated with the plurality of output ports 504.

The three optical switches 506, 508, 514 are interfaced by an optical junction 516. The primary function of the optical junction 516 is to route the optical signals to the appropriate destination. This generally means that source signals are routed into the catheter and the reflectances returning from the catheter are routed to the output ports. A secondary function of the optical junction 516 is to prevent optical signals from traveling to undesirable destinations. This generally means that the reflectances are prevented from reaching the input ports 502 and the source signals are prevented from directly reaching the output ports 504. These two functions are realized by implementing the optical junction with an optical circulator; however those skilled in the art will recognize that the optical junction can be built from combinations of other optical components, including wavelength-selective splitters (dichroic filters).

A controller 518 coordinates the operation of the three optical switches 506, 508, 514 so that the reflectances of an input signal of a certain type or wavelength are directed to the appropriate detector for analysis. This is facilitated by software routines processed by the controller 518 and commands received from an optional user interface 520. If required, the optical junction can also be placed under the control of the controller 518.

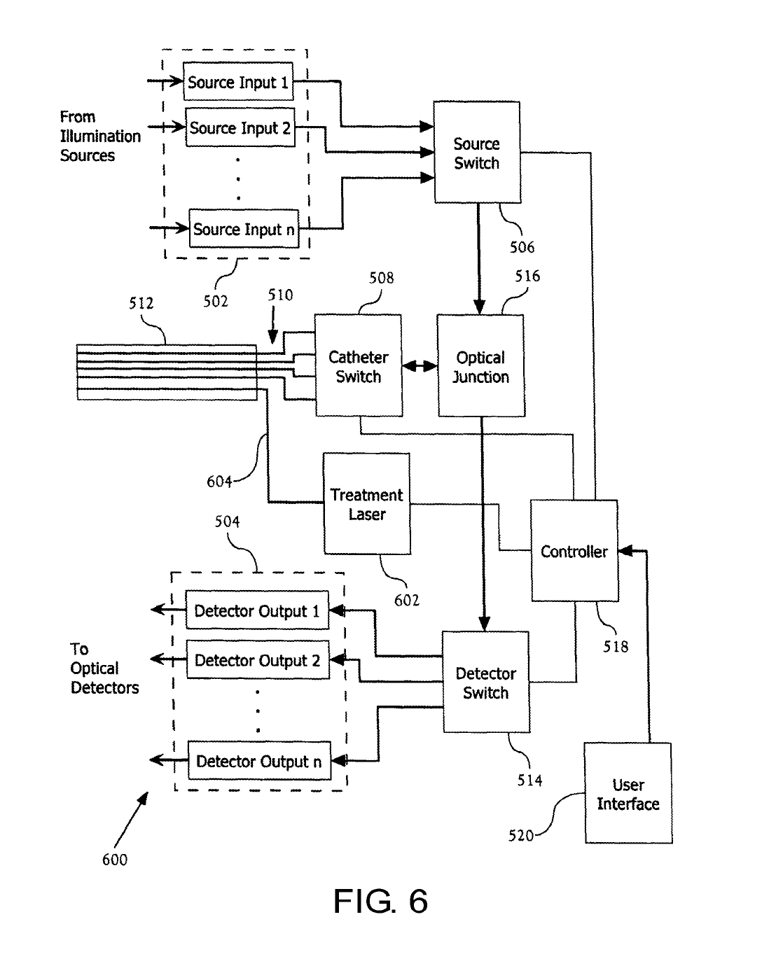

FIG. 6 illustrates still another embodiment of the medical apparatus 500 adapted for optical navigation, identification and treatment. This embodiment expands upon that shown in FIG. 5 with the inclusion of a treatment laser 602 and another optical fiber 604 in the catheter 512 for carrying the high power bursts of the treatment laser 602. The operation of the treatment laser 602 is coordinated in the system by the controller 520. Generally, during the operation of the treatment laser 602, any or all of the other optical switches are moved to a safe position to optically isolate the optical sources and detectors from potentially harmful back-reflections of the treatment laser 602. The safe position could be any position if the optical circulator provides optical isolation or can be a special position which connects the optical fibers 510 of the catheter 512 to optical dead-ends.

It should be noted that while the illustrated embodiments of FIGS. 1, 4, 5, and 6 show all three optical switches used together, the use of a single source switch, a single detector switch, and the various sub-combinations of the three switches are also contemplated by the present invention.

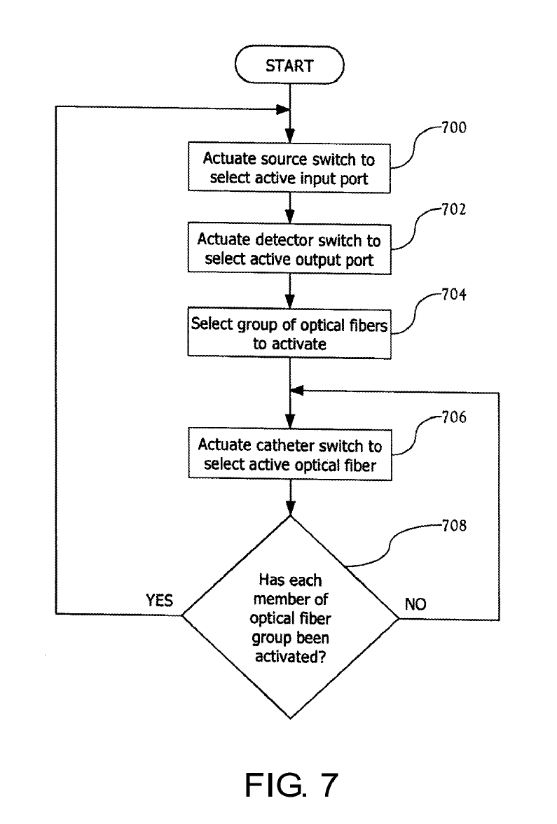

Another feature of the present invention is the ability to control the routing of the optical sources through the catheter to obtain a full picture of the lumen. By sending the signal from each optical source through a selected group of the optical fibers in the catheter a more accurate picture of the lumen is obtained. FIG. 7 is flow chart of the sequencing of the optical sources relative to the optical fibers in the catheter. First, the controller actuates the source switch 700 making a selected input port active so that signals from a desired source can be used. The controller also actuates the detector switch 704 making a selected output port active so that reflectances from the input signals are routed to the desired detector. A group of optical fibers is selected 706. This selection can be static, i.e., the same every time, or exhibit variability based upon detected conditions or user control. It is common for the group to include each optical fiber; however, subsets of the optical fibers can be selected. Next, the controller actuates the catheter switch to select the active optical fiber 706. This continues until each optical fiber in the group has been used 708. Those skilled in the art will recognize the activation sequence of the optical fibers can be varied without departing from the scope and spirit of the present invention.

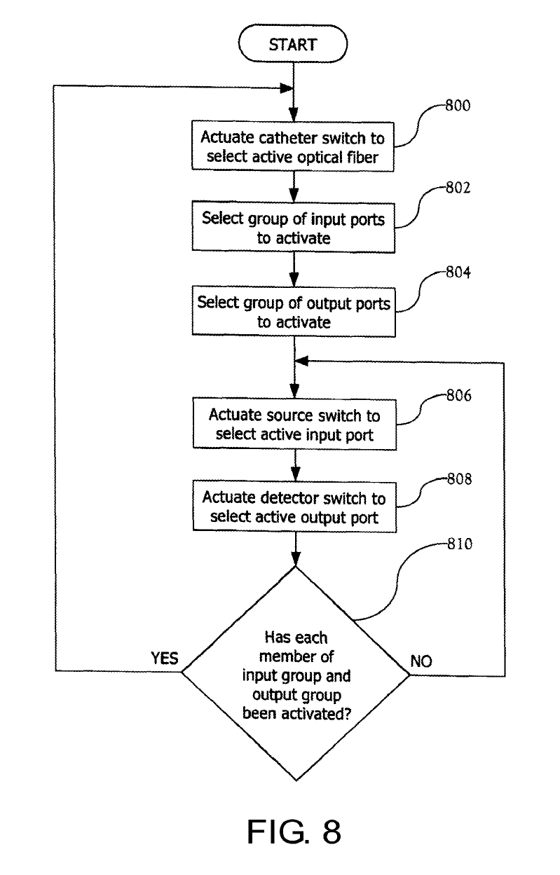

FIG. 8 is a flowchart of a variation on the sequencing function shown and described in reference to FIG. 7. In this variation, the active optical fiber in the catheter remains constant while the input ports and the corresponding output ports are rotated. First, the controller selects the active optical fiber in the catheter 800. Next, the group of input ports associated with the desired sequence of input sources is selected 802. This is followed by the selection the group of output ports associated with the desired optical detectors 804. Note that the input sources and detectors need not follow a one-to-one correspondence, as the reflectances from a single input source may be used by multiple optical detectors. The controller actuates the source switch to cycle through the selected group of input ports 806. The controller also actuates the detector switch to cycle through the selected group of output ports 808. The source switch and detector switch actuation continues until all selections of the input port group and the output port group have been made active 810.

For side/lateral-viewing catheter embodiments, any suitable sort of side/lateral-viewing optical assembly(ies) may be used and numerous sorts of side-viewing optics are known in the art. For example, a 45-deg (or other angle) mirror face or a prism can be used to laterally direct/redirect light from an optical fiber. Similarly, an optical fiber can be provided with an angularly faceted tip to direct and receive light that is off-axis with respect to the fiber. In one embodiment of the invention, the catheter is a basket-style optical catheter including at least two rod elements, such as 3, 4, 6 or 8, having side-viewing optical assemblies, each having a different radial field of view. Each side-viewing optical assembly may be associated with at least one optical fiber that longitudinally spans the body of the catheter from its proximal end to the side-viewing optical assembly so that light transmitted from the proximal end of the catheter, such as from a laser, can be directed to the blood vessel wall and light received from the blood vessel wall can be transmitted out of the catheter for analysis. U.S. Publication No. 2004/0260182 discloses intraluminal spectroscope devices with wall-contacting probes, and is incorporated by reference herein in its entirety. U.S. Publication No. 2005/0165315 discloses a side-firing fiber-optic array probe, and is incorporated by reference herein in its entirety.

The usefulness of the information obtained is largely dependent upon the acquisition speed of the information. A rapid acquisition speed allows both navigation and identification information to be obtained about the same location in the artery. If the acquisition speed is too low, the navigation information and the identification information are not associated with the same location within the artery and do not provide a complete picture. Obviously, the switching speed is dependent upon the forward movement speed and/or the rotational speed of the catheter and the number of wavelengths required to obtain a complete picture. The optical switching system of the present invention is capable of operating at the necessary switching speed to obtain useful information.

Certain characteristics of the optical switching system are useful in providing an efficient implementation; however, those skilled in the art will recognize that these characteristics are intended to be exemplary and not limiting. In various embodiments, the optical switching system exhibits low optical loss, nominally less than 1 dB, and low port to port variability, nominally less than 0.5 dB. The optical switching system latches in all positions, making the switch stable, resistant to shock and vibration and unintentional switching. The optical switching system exhibits temperature-independent operation with regard to optical performance. The optical switching system exhibits low polarization dependent loss, nominally less than 0.2 dB. The optical switching system exhibits a switching time quicker than 100 milliseconds.

Various aspects of the invention and embodiments thereof are further described below.

Optical analytical techniques that may be used alone or in the systems and methods of the invention include but are not limited to Raman spectroscopy, fingerprint region Raman spectroscopy, high-wavenumber region Raman spectroscopy, laser speckle spectroscopy, laser-induced fluorescence spectroscopy (LIFS), time-resolved LIFS, non-time resolved LIFS, absorption spectroscopy, near-infrared spectroscopy, near infrared absorbance spectroscopy, interferometry(ies), low coherence interferometry, optical coherence tomography (OCT), time domain OCT, and frequency domain OCT. While various examples in this disclosure are given with respect to particular combinations of optical analytical techniques in a system according to the invention, it should be understood that any and all combinations, as well as the general principle of operation of such combination(s) in a system according to the invention, are hereby disclosed and within the scope of the invention. An advantage of HW Raman spectroscopy is that the excitation and collected light in the catheter can be routed through the same optical fiber, simplifying combination with other measurement modalities, such as TR-LIFS, OCT, and LCI. Typical implementations of fingerprint Raman spectroscopy require the illumination and collection fibers to be physically separated in the catheter.

In one aspect, the apparatus of the invention may provide one or more different light sources, each light source being selected to provide the illumination for optical interrogation of a sample by a particular technique, such as a type of interferometry or spectroscopy. Lasers may, for example, be used as light sources for Raman spectroscopy, laser speckle spectroscopy, and laser-induced fluorescence spectroscopy, including time-resolved and non-time resolved methods. For various types of interferometry that utilize broad band light, light-emitting diodes (LEDs) may be used as light sources The invention also provides that, even with respect to a particular type of spectroscopy, more than one light source may be used such as for example with Raman spectroscopy where the preferred excitation wavelengths for analyzing light shifted into the fingerprint region is different than that for analyzing light shifted into the high wavenumber region. Similarly, for laser-induced fluorescence, such as time-resolved laser-induced fluorescence analysis, it may be desirable to use different light sources, such as ultraviolet lasers, having different wavelengths depending on the excitation wavelengths of various fluorescent chromophores of interest in a sample.

In a system according to the invention in which more than one light source is present, illumination by a particular light source may be selected using an N×1 optical switch, such as switch 104 shown in FIG. 1. Switch 104 may also be used to cycle between different light sources in order to cycle between different optical interrogation techniques, such as Raman spectroscopy, laser-induced fluorescence, laser speckle and interferometry-based techniques such as OCT.

Irrespective of whether the system has more than one different light sources for different optical analytical techniques or is operating using one or more different such light sources, there are various manners according to the invention by which the illumination from the light source(s) may be launched into the optical catheter.

The optical catheter may include one or more optical fibers in which light from the light source(s) is delivered to the sample. FIG. 1 illustrates a catheter having four optical fibers. Each optical fiber may terminate in an illumination assembly that has a different field of illumination as the next fiber. For example, in the case of a basket style optical catheter, each optical fiber may enter a different arm of the basket section of the catheter so that each fiber has a different radial field of illumination as the next.

A circulator, such as circulator 106, may be employed when the same fiber or fibers of the catheter are used for illumination of the sample and collection of light for analysis. With use of the circulator 106, light from the light source selected by switch 104 enters the circulator along path (port) 122 and exits circular 106 toward the catheter apparatus along path (port) 124. Light that has been collected from the sample returns to circulator 106 along path (port) 24 and is routed out of the circulator along path (port) 128 toward the optical detector(s)/analyzer(s).

A dichroic mirror may be used in some embodiments in place of a circulator. In this case, the dichroic mirror is selected to either transmit light from the light source(s) and reflect light from the catheter (from the sample) or alternatively reflect light from the light source(s) toward the catheter and transmit light from the catheter toward the optical detector(s)/analyzer(s).

If the system is configured with “parallel” illumination and collection fibers, neither a circulator nor a dichroic mirror is required. In this case, the optical paths leading to illumination of the sample and collection of light from the sample for transmission to the optical detector(s)/analyzer(s) may be completely separate.

In embodiments in which the catheter includes multiple optical fibers, such as those illuminating and/or collecting light from different fields of illumination/view, light may be handled in different manners with respect to the multiple fibers.

In the variation of the invention shown in FIG. 1, switch 110 sequentially controls which of the multiple (shown as four) optical fibers receive light from the light source. In this manner, where each fiber (or each pair of illumination and collection fibers) has a different field of view, a sequential scan of the fields of view may be performed. The fields of view may be different fields of view along the circumference of the artery lumen.

In another variation, multiple paired sets of illumination and collection fibers are provided in a similar manner, in which each pair may have a different field of view, such as a different radial field of view. In this variation, switch 110 is not present. Instead, illumination from the light source is either provided directly from the output of switch 104 or from path (port) 124 of the circulator (if present) and is split into multiple beam paths corresponding to each of the fiber pairs of the catheter using a beam splitter, such as a fused biconic beam splitter. In this manner, light from the light source is simultaneously directed down each of the different fiber paths of the catheter toward the sample. Light collected from the sample is directed down the collection fibers of the fiber pairs and may be handled in different ways. If the region-specific information corresponding to the different fields of view of the fiber pairs is important, it may be maintained. For example, with respect to analysis of the collected light by a spectrometer, light from the plural number of collection fibers can be separately imaged through one or more dispersion elements, each of which may be a diffraction grating, onto a detector array such as a CCD camera. If the region-specific information is not considered important or is not desired, light from the multiple collection fibers may be combined prior to entering the spectrometer, for example, by operating a splitter such as a fused biconic splitter in reverse to combine input from multiple fibers, or the imaging of the multiple fibers on the detector array may overlap, or the imaging of the multiple fibers on the detector array may be separate but the data obtained (for each wavelength range of interest) may be binned together.

As shown in FIG. 1, if the system includes more than one light detector/analyzer element (box 122), a switch 112 may be provided to control which of the light detectors/analyzers receives light from the catheter (from the sample). In variations in which circulator 106 is present, switch 112 will receive light from the catheter via path (port) 128 of the circulator. In variations that include separate illumination and collection fibers, switch 112 may receive light from the catheter (from the sample) directly form the collection fiber(s) or otherwise without the intermediate use of a circulator.

Spectrometers used for the various embodiments of the invention and for various types of spectroscopy may generally include a wavelength dispersion element such as a diffraction grating and a detector array such as a CCD camera. Since different optical analytical techniques that may be used together in a system according to the invention, such as fingerprint region and high-wavenumber region Raman spectroscopy and time-resolved ultraviolet laser-induced fluorescence spectroscopy may produce divergent ranges of optical signals, it is not always practical or cost-effective to use a single, statically configured spectrometer for the different optical analytical techniques. Instead, for example, the system may include separate spectrometers for different optical analytical techniques where it is impractical to use a single spectrometer. In this case, a switch, such as switch 112, may be used to select which spectrometer receives light from the sample. Alternatively, a spectrometer that is configurable to the wavelength range and resolution requirements of the various optical analytical techniques may be used. For example, one embodiment of the invention provides a configurable spectrometer that includes a common detector array such as a CCD camera but separate dispersion elements such as separate diffraction gratings that may be switched in or out of use accordingly, for example, by rotation of a wheel on which the diffraction gratings are mounted. Another embodiment of a configurable spectrometer that may be used includes a common detector array such as a CCD camera and a dispersion element such as a diffraction grating that is mounted on a positional control assembly so that the angle and/or relative position of the dispersion element and detector area can be selectively configured for different optical analytical techniques.

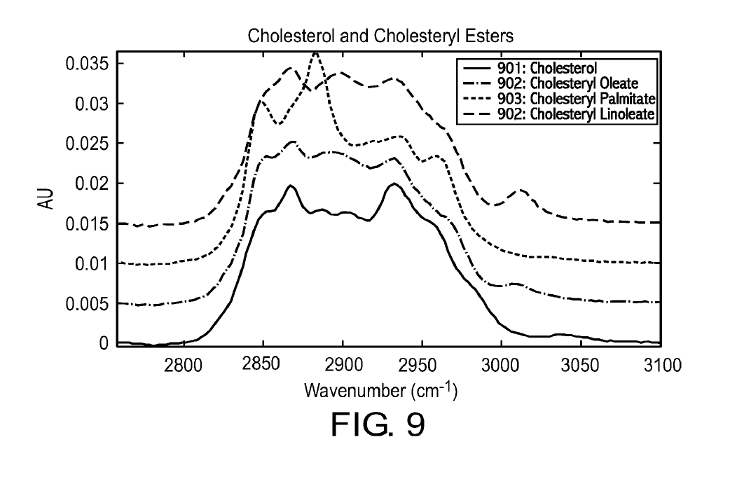

The ability of the apparatuses and systems of the present invention to switch and cycle between high wavenumber Raman spectroscopy and other optical analytical techniques, such as fingerprint region Raman spectroscopy, is advantageous in the in situ analysis of biological tissue, especially in the evaluation of health and disease of the vasculature. FIG. 9 shows Raman spectra of cholesterol and cholesterol esters collected using free space optics in the high wavenumber region. Specifically, curve 901 is a Raman spectrum for cholesterol, curve 902 is a Raman spectrum for cholesterol oleate, curve 903 is a Raman spectrum for cholesterol palmitate and curve 904 is a Raman spectrum for cholesterol linolenate.

A further embodiment of the invention provides catheter-based spectroscopic systems that are configured to cycle between measuring Raman scattered light in the high-wavenumber region, i.e., 2,500-4,000 cm⁻¹, such as in the range 2,500-3,700 cm⁻¹, and performing another optical analytical technique, such as measuring Raman scattered light in the fingerprint region, i.e., 200-2,500 cm⁻¹, such as within the range 400-2,500 cm⁻¹, and related methods. Still another aspect of the invention provides systems and related methods that involve cycling between Raman spectroscopy (fingerprint and/or high wavenumnber) and time-resolved laser-induced fluorescence spectroscopy. In these cases, the laser source(s) may be of any suitable kind for the Raman excitation and fluorescence excitation of the various embodiments of the invention. The laser source may, for example, be a diode-pumped solid state (DPSS) laser. The laser source may, for example, be a feedback-stabilized multi-mode laser diode such as a volume Bragg grating (VBG)-stabilized multi-mode laser diodes (available from PD-LD Inc., Pennington, N.J.) or single mode lasers, such as those known in the art. In one implementation of the invention, a separate feedback-stabilized multi-mode laser diode is used for each illumination/excitation wavelength required.

Design considerations for Raman spectroscopy fiber optic probes are discussed in Motz et al. (2004) Optical Fiber Probe for Biomedical Raman Spectroscopy, Applied Optics 43(3): 542-554, which is incorporated by reference herein in its entirety. For fingerprint region Raman spectroscopy, delivery fiber(s) may be terminated with a short-wavelength-pass or a bandpass filter that transmits the laser excitation light while blocking the longer-wavelength spectral background from the fibers. The collection fiber(s) may be preceded by a long wavelength-pass filter or notch filter, which transmits the sample's Raman spectrum while blocking laser light backscattered from the tissue. However, as pointed out in U.S. Publication No. 20060139633, high wavenumber Raman spectroscopy does not require such filters and, therefore, a single optical fiber may be used for both illumination and collection.

One embodiment of a Raman spectroscopy system according to the invention that cycles between fingerprint region Raman spectroscopy and high-wavenumber Raman spectroscopy may be described as a variation of the system shown in FIG. 1. In this embodiment, there are two lasers, Laser 1 emitting at or around 785 nm for fingerprint region measurements and Laser 2 emitting at or around 671 nm for high-wavenumber region measurements. Laser 1 may, for example, be a spectrum stabilized 785 nm Laser P/N#: 10785SL0080PA from Innovative Photonics Solutions (Monmouth Junction, N.J., USA). Laser 2 may, for example, be a Model RCL-100-671 100 mW, 671 nm, TEMoo, DPSS, CW laser with power supply from CrystaLaser (Reno, Nev., USA). In this embodiment, the optical detectors may, for example, consist of a single Raman spectrometer for detecting wavenumber-shifted light from a sample illuminated by the lasers. In this case, switch 112 may be eliminated of there are no other types of detectors used for other types of optical analytical techniques. The single spectrometer element may, for example, include a HPRM2500 High Performance Raman Module spectrograph from River Diagnostics (Rotterdam, The Netherlands) and an InDus CCD: e2V CCD40-11. 1024×125, 26 mm² CCD camera from Andor (South Windsor, Conn., USA). Alternatively, a separate spectrometer element may be provided to detect Raman scattered light in the fingerprint region and in the high-wavenumber region. The embodiment may be configured with or without switch 110. Generally, a separate excitation and collection fiber are needed for fingerprint region Raman spectroscopy. A separate optical fiber for performing illumination and collection for high-wavenumber region Raman spectroscopy may be provided or one of either the fingerprint Raman illumination or collection fibers may be used to perform high wavenumber Raman spectroscopy. For instance, a band blocking filter could be placed at the distal end of the collection fiber for fingerprint Raman scattered light collection, said filter blocks the Rayleigh scattered fingerprint excitation light from entering the fiber yet passes the excitation light for high wavenumber Raman spectroscopy onto the tissue and is capable of collecting the resulting scattered light in the fingerprint and high wavenumber regions. The high wavenumber and fingerprint regions may overlap if different excitation lasers are chosen appropriately for the two forms of spectroscopy.

A related embodiment provides a system, such as an intravascular catheter-based system, configured or configurable to switch or cycle between fingerprint region and high wavenumber region Raman spectroscopy which may be configured in the manner of FIG. 5 but provide one or more fingerprint Raman collection optical fibers that bypass catheter switch 507 and optical junction 516 and connect directly, or just via a switch, to a suitable detector element, i.e., a Raman spectrometer. In this case, excitation for fingerprint Raman spectroscopy may be provided from a laser source in 502 and travel thorough the illumination path shown in FIG. 5 to a sample, while inelastically scattered light received from the sample is delivered to the Raman spectrometer by the separate collection fiber(s). A separate laser source may be used in 502 for high wavenumber Raman spectroscopy. For high wavenumber Raman spectroscopy, the same optical fiber is used for excitation and collection and inelastically scattered light resulting from illumination of a sample is collected by the same fiber used for excitation and transported back through switch 508 and junction 516 toward a suitable detector, i.e., a Raman spectrometer.

A further related embodiment provides a system, such as an intravascular catheter-based system, configured or configurable to switch or cycle between fingerprint region and high wavenumber region Raman spectroscopy may be configured in a manner similar to the system shown in FIG. 6 but where a laser source for fingerprint Raman spectroscopy is placed in a similar position as treatment laser 602, having input into the fingerprint Raman illumination fiber(s) of the catheter probe under the control of controller 518. In this case one or more fingerprint Raman collection optical fibers may be connected to catheter switch 507 so that collected light can be directed into optical junction 516 and from there be directed to a suitable detector, i.e., a Raman spectrometer, in the manner shown. A separate laser source may be used in 502 for high wavenumber Raman spectroscopy. As previously pointed out, for high wavenumber Raman spectroscopy, the same optical fiber is used for excitation and collection and inelastically scattered light resulting from illumination of a sample is collected by the same fiber used for excitation and transported back through switch 508 and junction 516 toward a suitable detector, i.e., a Raman spectrometer.

A related embodiment of the invention further provides time-resolved laser-induced fluorescence spectroscopy (TR-LIFS). Thus, the fingerprint region and/or high-wavenumber region systems described above may further include a third laser, Laser 3, emitting in the ultraviolet region, for example, at or near 337 nm. A different spectrometer/spectrum analyzer may be provided than that/those used for the analyzing the Raman scattered light, in which case switch 112 may be operated to select between the detectors. The same and/or separate optical fibers in the catheter may be used to deliver the laser light and collect light from the sample for delivery to the optical detectors. In the case that different optical fibers are used, switch 110 may be operated to select the fibers.

A system according to the invention may be configured to cycle between types of interferometry and/or spectroscopy that the system is capable of, in different manners. In one variation, the system is capable of operating in different modes which can be selected by the user, for example to perform any one of the optical analytical techniques that the system is capable of or to cycle between various combination(s) of the techniques as the catheter interrogates a blood vessel or other lumen. By way of example, for a system that is capable of performing fingerprint region Raman spectroscopy, high-wavenumber region Raman spectroscopy, in one mode, the system cycles between fingerprint region Raman spectroscopy, high-wavenumber Raman spectroscopy and TR-LIFS. In another mode, the system cycles between either of Raman spectroscopy, and high-wavenumber Raman spectroscopy, and TR-LIFS. In another mode, the system cycles between fingerprint region Raman spectroscopy and high-wavenumber Raman spectroscopy, but does not perform TR-LIFS. In another mode, the system performs either fingerprint region Raman spectroscopy or high-wavenumber Raman spectroscopy, but does not perform TR-LIFS. In still another mode, the system performs only TR-LIFS and not Raman spectroscopy.

One embodiment of the invention provides a catheter-based Raman spectroscopy system, such as for evaluating blood vessels, that includes: a catheter; one or more optical fibers carried by said catheter; one or more optical inputs such as a plurality of optical inputs; one or more optical detector outputs such as a plurality of optical detector outputs; at least one Raman spectrometer for measuring Raman scattered light from a tissue sample; and at least one laser source for illuminating a tissue sample, wherein the system is configured to cycle between measuring Raman scattered light in the fingerprint region (200-2,500 cm⁻¹) and high wavenumber region (2,500-4,000 cm⁻¹). The system of may further include an analyzer comprising at least one microprocessor and configured for diagnosing the tissue sample based on the measurement by the spectrometer of Raman scattered light from the sample in the fingerprint region and in the high-wavenumber region. Generally, the analyzer includes at least one microprocessor working in conjunction with computer memory under the control of computer instructions, such as software.

In one variation of the embodiment, the system includes: a first laser source providing excitation illumination of a first wavelength for stimulating Raman scattered light emissions from the tissue sample in the fingerprint region; a second laser source providing excitation illumination of a first wavelength for stimulating Raman scattered light emissions from the tissue sample in the high wavenumber region, wherein the first wavelength and second wavelength are different; and an optical switch configured to control which of the first and second laser sources provides illumination to the tissue sample and to cycle between illumination by the first and second laser sources. The first wavelength (and wavelength generally for fingerprint region Raman) may be at or about 785 nm and the second wavelength (and wavelength generally for high-wavenumber Raman) may be at or about 671 nm.

In a different variation of the embodiment, the system comprises a single laser source emitting light at or around a single wavelength; and the at least one Raman spectrometer comprises separate detection elements for detection of Raman scattered light in the fingerprint region and in the high-wavenumber region. This variation may include an optical switch configured to control which of the separate detection elements receives Raman scattered light from the tissue sample and to cycle between the separate detection elements. Alternatively, Raman scattered light from the tissue sample is simultaneously directed onto the separate detection elements. In this, latter case, the system may further include a fiber splitter configured to divide the Raman scattered light from tissue sample (from the output fiber(s)) into more than one separate optical paths among/from which different optical paths are directed onto the separate detection elements.

In still another variation of the embodiment, the system is further configured to perform time-resolved laser-induced fluorescence spectroscopy and to cycle between taking the Raman spectroscopy measurements (either one or both of fingerprint and high-wavenumber) and time-resolved laser-induced fluorescence spectroscopy (TR-LIFS) measurements of the sample. In this variation, the system may include a separate laser source (from the one or more Raman excitation laser sources) for fluorescent excitation of the sample. In this case, the optical switch will cycle between the one or more Raman laser sources and the fluorescence excitation laser source. The separate laser source for fluorescence excitation of the sample may have an emission wavelength in the ultraviolet range such as at or about 337 nm. The analyzer of the system may be configured to analyze both the Raman spectroscopy data and the TR-LIFS data to provide a diagnosis of, or other indication of, the condition of a blood vessel.

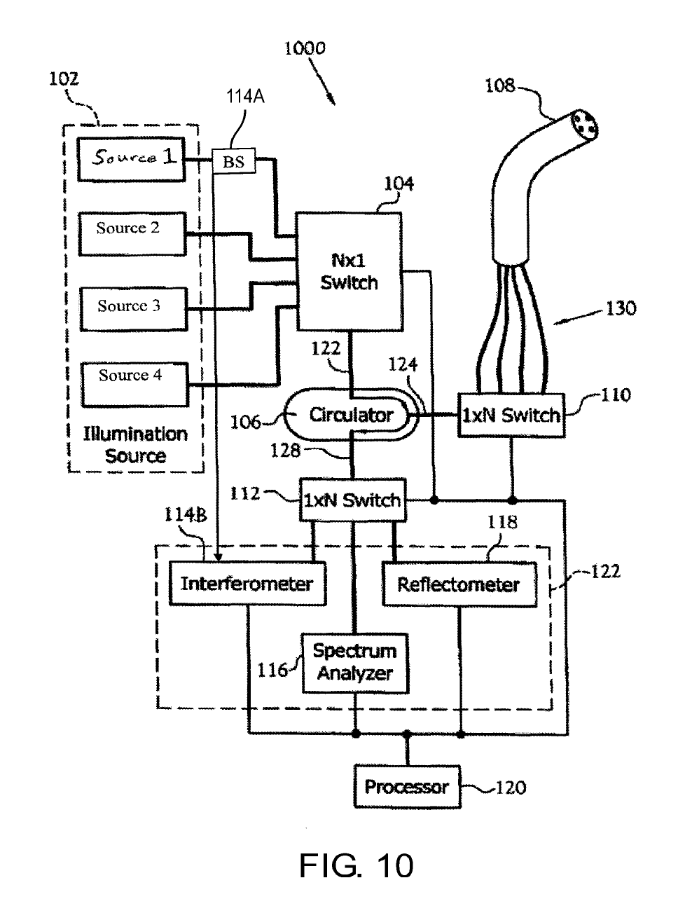

FIG. 10 shows an embodiment 1000 similar to that of FIG. 1, but which includes a particular configuration for performing interferometry, such as any type of OCT. In FIG. 10, the first illumination source is low-coherence light source, such as a broadband light source, such as a super-luminescent LED for performing interferometry. A beam splitter (BS) 114A transmits a portion of the light from source 1, such as 50%, as a reference beam towards a mirror that is part of the interferometer and transmits a portion of light, such as 50%, toward the sample, via switch 104, as a sample beam. The reference and sample beams are combined in interferometer element 114B. Thus, 144A and 114B collectively constitute a Michelson interferometer. The system may cycle through or switch between interferometry and any other analytical technique that the system is capable of, as previously described.

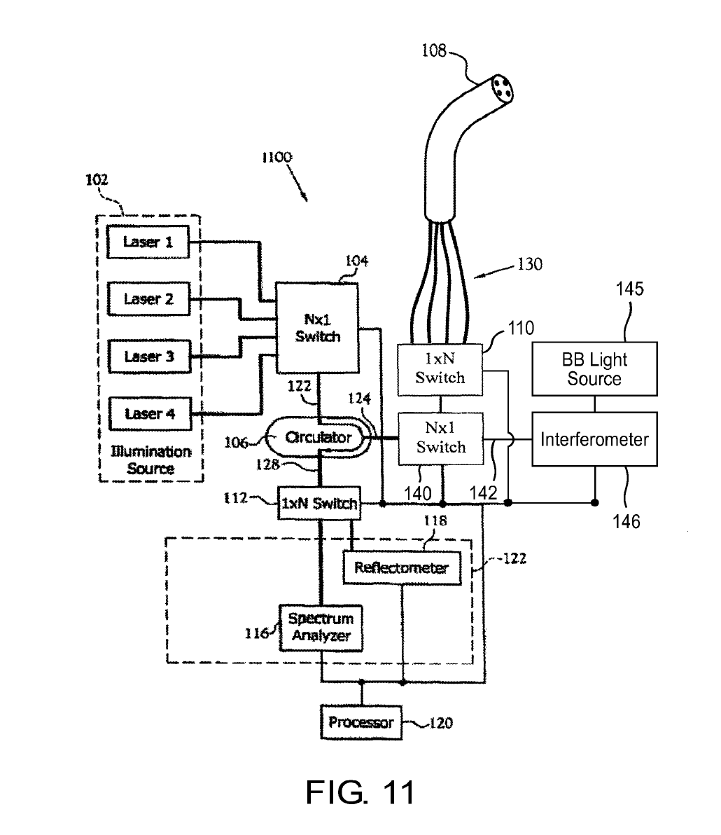

FIG. 11 shows an embodiment 1100 similar to that of FIG. 1 except that an N×1 switch 140 has been added between circulator 106 and 1×N switch 110 so that an interferometer module including a low-coherence, broadband (BB) light source 145 and an interferometer 146, can be more compactly and simply integrated with the system. The broadband light source 145 may, for example be a super-luminescent LED. The interferometer 146 may, for example, be a Michelson-type interferometer. The photodetector(s) for interferometer 146 are not separately shown, but are part of the interferometer unit 146 in the figure. Data from the interferometer unit is analyzed by processor 120. Switch 140 is operated to control whether light will be received and sent along path 124 from/to circulator 106 to perform analytical techniques corresponding to illumination sources 102 or whether light will be received and sent along path 142 to perform interferometry techniques such as OCT using the interferometry module. Switch 110 may be operated in a similar manner as previously described. In one variation of the embodiment, not shown, a treatment laser can also be provided. The output of the treatment laser could, for example, be directed into switch 140 and distributed to optical fibers 130 by switch 110. In an alternative, the output of the treatment laser could, for example, be directed down one or more entirely separate treatment optical fibers disposed within the catheter, similar to the embodiment shown in FIG. 6.

In the figures, Lasers 1 through 4 are shown for illustration only; the illumination sources 102 may generally include one or more light sources which may or may not be lasers. For the sake illustration: Laser 1 could, for example, be a laser for fingerprint region Raman spectroscopy; Laser 2 could, for example, be a laser for high-wavenumber region Raman spectroscopy; Laser 3 could, for example, be an ultraviolet (UV) laser for performing LIFS, such as TR-LIFS; and Laser 4 could, for example, be a near infrared laser for performing any type of NIR spectroscopy.

The optical switches used in various embodiments of the invention may be of any suitable kind For example, bulk optical approaches such as electrical relay-controlled prisms may be used. Acousto-optical switches may be used and permit nano-second scale switching speeds, Acousto-optical switching is disclosed, for example in U.S. Pat. No. 6,922,498. Micro-electromechanical system-based (MEMS) optical switches may also be used, such as those involving the positioning of micro-mirrors and are disclosed, for example, in U.S. Pat. No 6,396,976. Bubble-based optical switching mechanisms that involve the intersection of two waveguides so that light is deflected from one to the other when an inkjet-like bubble is created may also be used and are disclosed, for example, in U.S. Pat. No. 6,212,308. Electro-optical switches of various types may also be used. One type of electro-optical switch employs the electro-optical effect of some materials in which the index of refraction changes under the influence of an applied electrical field. Such materials include lithium niobate, electro-optical ceramics, polymers and other nonlinear optical and semiconductor materials. The materials may be incorporated into an arm of an interferometer to control the propagation direction of light. Fast switching times can be obtained with electro-optical switches, on the order of nanoseconds for lithium niobate. Operation and coordination of the various switches of embodiments of the invention and for the various modes of operation thereof may be under the control of one or more microprocessors and/or control circuits. The beam splitters used in various embodiments of the invention may be of any suitable kind For example, fiber optic beam splitters may be used, such as fused biconic fiber optic beam splitters.

From the foregoing description, it will be recognized by those skilled in the art that a device and method for safely navigating blood vessels using a catheter has been provided. The device and method uses an optical switch to control the inputs and outputs of optical fibers set in a catheter. The device can differentiate among arterial walls, calcified plaque, vulnerable plaque (biological hot plaque and thin capped fibrous atheromas), and other forms and substances in blood vessels. The device is useful in the treatment of the arteries of the heart, Atherosclerosis, Arteriosclerosis, Thrombosis, for the performance of Hemodialysis Access Maintenance, and for the insertion of Transjugular Intrahepatic Portosystemic Shunts. In addition, the device provides for the intermittent or concurrent use of a treatment laser, such as an excimer laser, or other treatment tool, such as a stent or an angioplasty balloon, in conjunction with one or more interferometer systems and devices by use of optical switches.

The invention also provides methods for evaluating the condition of blood vessels using the optical catheter system embodiments of the invention, which may, for example, include identifying, locating and/or characterizing atherosclerotic lesions in a blood vessel, such as an artery, using any system embodiments of the invention. A related embodiment of the invention provides methods for identifying, locating and/or characterizing vulnerable plaque lesions in a blood vessel, such as an artery, using any of the system embodiments of the invention. One embodiment of the invention is a method for diagnosing and/or locating one or more vulnerable plaque lesions in a blood vessel, such as a coronary artery of a subject, using a system as described herein to optically evaluate the properties of a vessel wall at one more locations along the vessel. Still another related embodiment of the invention provides methods for identifying, locating and/or characterizing lipid-rich deposits or lesions in a blood vessel, such as an artery, using any of the system embodiments of the invention. Any of the methods of evaluating the condition of a blood vessel using a catheter system according to the invention may include moving the catheter laterally within a blood vessel to optically interrogate the blood vessel wall at different lateral positions. Optical sampling of the vessel wall may be performed while the optical catheter is moving laterally within a blood vessel and/or while it is stopped at a lateral position within the vessel.

Each of the patents and other publications cited herein is incorporated by reference in its entirety.

While the present invention has been illustrated by description of several embodiments and while the illustrative embodiments have been described in detail, it is not the intention of the applicant to restrict or in any way limit the scope of the appended claims to such detail. Additional advantages and modifications will readily appear to those skilled in the art. The invention in its broader aspects is therefore not limited to the specific details, representative apparatus and methods, and illustrative examples shown and described. Moreover, features described in connection with one embodiment of the invention may be used in conjunction with other embodiments, even if not explicitly stated above. Accordingly, departures may be made from such details without departing from the spirit or scope of applicant's general inventive concept.

What is claimed is:

1. A catheter-based optical analytic apparatus capable of performing more than one optical analytical technique, comprising:
a catheter;
one or more optical fibers carried by said catheter to deliver light to a sample and receive light from the sample;
at least two light sources, wherein at least one light source is selected to provide illumination for high-wavenumber region (2,500-4,000 cm⁻¹) Raman spectroscopy and at least one light source is selected to provide illumination for at least one other optical analytical technique; and
a first switch operable to select which of the light sources provides light to the catheter,
said apparatus being configured or configurable to cycle between performing Raman scattered light high-wavenumber region (2,500-4,000 cm⁻¹) during a catheter procedure and performing the at least one other optical analytical technique.

2. The apparatus of claim 1, wherein the at least one other optical analytical technique comprises fingerprint region (200-2,500 cm⁻¹) Raman spectroscopy.

3. The apparatus of claim 1, wherein the at least one other type of optical analytical technique consists of fingerprint region (200-2,500 cm⁻¹) Raman spectroscopy.

4. The apparatus of claim 1, wherein the at least one other optical analytical technique comprises laser-induced fluorescence spectroscopy.

5. The apparatus of claim 4, comprising an ultraviolet laser as one of the light sources for performing laser-induced fluorescence spectroscopy.

6. The apparatus of claim 5, wherein the ultraviolet laser has an emission wavelength at or about 337 nm.

7. The apparatus of claim 4, wherein the laser-induced fluorescence spectroscopy comprises time-resolved laser-induced fluorescence spectroscopy.

8. The apparatus of claim 1, wherein the at least one other optical analytical technique comprises interferometry.

9. The apparatus of claim 8, wherein the interferometry comprises optical coherence tomography.

10. The apparatus of claim 9, wherein the apparatus further comprises a low-coherence light source for performing the interferometry.

11. The apparatus of claim 2, wherein the at least two light sources comprise a laser having an emission wavelength at or about 785 nm for fingerprint region Raman spectroscopy and a separate laser having an emission wavelength at or about 671 nm for high-wavenumber region Raman spectroscopy.

12. The apparatus of claim 1, further comprising:
at least one light analyzing element comprising at least one Raman spectrometer; and
a circulator positioned between the switch and the catheter, said circulator directing light from the first switch toward the catheter and directing light from the catheter toward at least one light analyzer element.

13. The apparatus of claim 1, wherein the catheter comprises a plurality of optical fibers and wherein the apparatus further comprises:
a second switch that selects which of the optical fibers receives light from a light source that is selected by the first switch.

14. The apparatus of claim 1, wherein the catheter comprises a plurality of optical fibers and the apparatus further comprises:
at least one light analyzing element comprising at least one Raman spectrometer;
a circulator positioned between the switch and the catheter, said circulator directing light from the first switch toward the catheter and directing light from the catheter toward at least one light analyzer element; and
a second switch that selects which of the optical fibers receives light from a light source that is selected by the first switch, wherein the second switch is positioned between the circulator and the catheter.

15. The apparatus of claim 2, wherein the apparatus comprises:
at least two light analyzer elements comprising at least two Raman spectrometers, one configured to measure light in the fingerprint region and one configured to measure light in the high wavenumber region; and
a switch operable to select which of the light analyzer elements receives light from the catheter.

16. The apparatus of claim 1, wherein the first switch has a nanosecond-order switching speed.

17. The apparatus of claim 2, wherein the apparatus is configurable to cycle between at least one of fingerprint region and high-wavenumber region Raman spectroscopy and at least one other optical analytical technique.

18. The apparatus of claim 2, wherein the apparatus comprises an illumination optical fiber for fingerprint Raman spectroscopy and a separate collection optical fiber for fingerprint region Raman spectroscopy and is configured or configurable so that illumination and collection functions for high wavenumber Raman spectroscopy are performed using the same optical fiber.

19. The apparatus of claim 18, wherein an optical fiber separate from the fingerprint Raman spectroscopy illumination and collection optical fibers is used for high wavenumber Raman spectroscopy.

20. The apparatus of claim 18, wherein the apparatus is configured or configurable to perform high wavenumber Raman spectroscopy over the fingerprint Raman spectroscopy illumination fiber or the fingerprint Raman spectroscopy collection fiber.

21. The apparatus of claim 1, wherein the catheter is an intravascular catheter.

22. A method for optically interrogating a blood vessel, comprising the steps of:
providing an apparatus according to claim 21;
inserting the catheter of the apparatus into a blood vessel; and
moving the catheter along the blood vessel as the apparatus cycles between performing high-wavenumber Raman spectroscopy and the at least one other optical analytical technique.
